(19) United States
(12) Patent Application Publication
(10) Pub. No.: US 2024/0099301 A1
(43) Pub. Date: Mar. 28, 2024

(54) Disinfecting and Sanitizing Composition, Method for Preparing the Composition and Use of Same

(71) Applicant: SOCIEDAD QUÍMICA ECOLÓGICA SPA, Coquimbo (CL)

(72) Inventor: Elmo Enrique RODRIGUEZ BARAHONA, Coquimbo (CL)

(73) Assignee: SOCIEDAD QUÍMICA ECOLÓGICA SPA, Coquimbo (CL)

(21) Appl. No.: 18/264,439

(22) PCT Filed: Jan. 10, 2022
(86) PCT No.: PCT/CL2022/050003

§ 371 Date: Aug. 7, 2023

(30) Foreign Application Priority Data
Feb. 25, 2021 (CL) 477-2021

(51) Int. Cl. A01N 43/16; A01N 25/06; A01N 59/20; A01N 65/22; A01N 65/28; A01P 1/00

(52) U.S. Cl. CPC A01N 43/16; A01N 25/06; A01N 59/20; A01N 65/22; A01N 65/28; A01P 1/00

(57) ABSTRACT

Disinfectant and sanitizing composition comprising a mixture of 1-4% w/v of a chitin derivative, such as the biopolymer chitosan, with molecular weight between 300.000 gr/mol and 600.000 gr/mol and viscosity between 48 and 54 mPa/s, 1-3% w/w of an organic acid, 0.002-0.01% w/v of copper nanoparticles, 1-2% v/v of a vegetable essential oil, and the remainder being water. Also disclosed are a method to prepare this composition and descriptions of its use.

### CROSS-REFERENCES TO RELATED APPLICATIONS

This Non-Provisional Application claims the benefit of and priority to PCT Application Serial No. PCT/CL2022/050003, filed Jan. 10, 2022, entitled “Disinfecting and Sanitising Composition, Method for Preparing the Composition and Use of Same,” which claims the benefit of and priority to Chilean Patent Application Serial No. CL 477-2021, filed Feb. 25, 2021, entitled “Disinfecting and Sanitising Composition, Method for Preparing the Composition and Use of Same,” the entire contents of both applications of which are hereby incorporated herein by reference.

### BACKGROUND

The present invention refers to a biodegradable and biocompatible sanitizing and disinfectant mixture that can be used on surfaces and people in different environments. Due to its characteristics, it can be applied in the presence of people, so its use is recommended in health establishments and all types of establishments open to the public. For that reason, it is an ideal product for sanitation tunnels, as it is harmless to people. The applications of the product extend to disinfection and general sanitation of utensils, furniture, equipment, walls and floor in various environments (such as schools, homes, industries and health facilities, among others).

Disinfection and sanitation of areas has always existed; the emergence of the COVID-19 Pandemic, however, made it is an even more prevailing and recurrent topic among various users.

People need to keep spaces free of contamination and avoid contagion as much as possible. Even in this context, a large part of the population does not know the products that are used for these purposes, and in some cases the products offered are not suitable for application on people, as happens with products applied in sanitation tunnels. There is a wide variety of solutions in the market that aim to disinfect and sanitize areas; however, many have some degree of toxicity and could be harmful to humans. Given the above, the need arises to innovate with an effective alternative disinfectant and sanitizer able to be applied for daily use in environments while people carry out their activities.

Disinfection is a process that inactivates contaminants such as bacteria, viruses and protozoa, preventing their development and growth on surfaces. Sanitization helps reduce the number of microorganisms by using liquids with antimicrobial properties applied, for example, using high-pressure treatments to produce a spray of nebulized liquid and eliminate particles or microorganisms. That makes clear that disinfectants are consequently sanitizing, only the dilutions (in the case of chemicals) and application forms changing between them.

Several forms of disinfection exist, depending on the place of application and purpose. The first known form are physical disinfection systems:

Heat: Dry or moist. Dry heat disinfection is performed by exposing objects to hot air inside an oven with temperatures between 160° C. and 170° C. (320-338° F.) and is very useful for bactericidal disinfection of glass and metal. Moist heat disinfection is used to disinfect instruments and objects in a liquid medium by using autoclaves. It is also used to disinfect water by heating it to the boiling point; that is, the water must reach a temperature of 100° C. (212° F.).

Incineration. Incineration destroys all life, and therefore also kills bacteria. This method can be used to destroy all kinds of materials and disinfect metals when they reach incandescence.

Filtration. Filtration is a sterilization system widely used in laboratories to disinfect substances that cannot be treated with heat. This method can isolate bacteria, spores and fungi, but does not work against viruses.

The field of cleaning and disinfection also employs equipment containing groups of antimicrobial agents to inhibit bacterial reproduction, including:

Filtration equipment: these devices contain bactericidal or bacteriostatic substances and are used as effective disinfectants in water and toilet hygiene; for example, there are activated carbon filters for water treatment that contain a certain amount of silver or copper and granulated zinc in their composition.

Ultraviolet radiation generators: also included in this group are purifying devices that generate ultraviolet rays that kill bacteria and other microorganisms in environments such as laboratories, food industry facilities or hospitals.

Bacteriostatic equipment: one application of bacteriostatic agents that is highly effective for cleaning and disinfecting toilets, especially those used by many different people, are the devices also known as “bacteriostatic toilet dispensers”, which, when installed, discharge into the toilet pan bactericidal substances that prevent the proliferation of bacteria. The application of bactericidal products reduces dirt retention, guarantees better conditions of use in toilets, and eliminates bad odors due to the decomposition of organic secretions. These devices can be activated either along with flushing or automatically at predetermined intervals.

Ozone equipment: an ozonator is a machine that artificially produces ozone for use in environmental disinfection and cleaning, odor elimination, water treatment and purification, and medical treatments. There are different types of ozonators, depending on their size, power, characteristics, and accessories.

Finally, there is the group of antimicrobial pesticides, which are substances or mixtures of substances used to destroy or suppress the growth of harmful microorganisms such as bacteria, viruses or fungi on inanimate objects and surfaces. These antimicrobial products contain different active ingredients and are marketed in various formulations: aerosols, liquids, concentrated powders, and gases.

A product with antimicrobial action is a substance or mixture of substances that disinfects, sanitizes, reduces or mitigates the growth or development of microbial organisms (bacteria, viruses, fungi, algae, protozoa, yeasts and others), protecting inanimate objects, industrial processes, surfaces, water or other substances or chemicals from contamination, deterioration, development of biofilms, among others. This action can be carried out by disinfection, sanitization, sterilization, deodorization and other similar processes.

This group includes disinfectant products, which contain chemicals that destroy or inactivate microorganisms that cause infections. Many chemical disinfecting agents exist, as detailed below:

Iodine and iodophors: iodine, especially in its molecular form, can penetrate microorganisms' cell walls and membranes quickly, which can be considered its fundamental characteristic. Iodine has proven to be an excellent germicidal agent. However, its corrosivity, poor solubility, strong smell, and tendency to dye the surfaces on which it is applied make it unusable in the agri-food industries. However, these drawbacks are neutralized when iodine is bound to a nonionic surface-active agent, which acts as a solvent and carrier of iodine, forming so-called iodophors. Their spectrum of action includes Gram-positive and Gram-negative bacteria, moulds, yeasts and viruses, but they are not very active against bacterial spores. Iodophors are generally used at concentrations of 10-100 ppm, at temperatures up to 50° C., or 122° F. (Perez, 2014).

Oxygen compounds (chlorine-free): the best-known and cheapest of these compounds is hydrogen peroxide. At room temperature, hydrogen peroxide is a colorless liquid with a bitter taste. Small amounts of gaseous hydrogen peroxide are found naturally in air. It is unstable and quickly decomposes to oxygen and water while releasing heat, which does not cause damage to the environment. Although not flammable, hydrogen peroxide is a potent oxidizing agent that can cause spontaneous combustion when in contact with organic matter. It is mainly used in liquid presentations for high level disinfection (HLD) and in gaseous forms for disinfection of surfaces in healthcare centers. Hydrogen peroxide has bactericidal, bacteriostatic or sporicidal activity, depending on its concentration and the conditions of use (bacteriostatic activity at 3% and bactericidal activity at 6%, at room temperature). Although hydrogen peroxide is not effective on intact skin by itself, it is used in combination with other antiseptics to disinfect hands, skin and mucous membranes. At concentrations above 20% it is corrosive and oxidizing. Another oxygen compound is peracetic acid, which is not as well-known as hydrogen peroxide, but is a disinfectant with greater power and wider spectrum of use. Peracetic acid is not corrosive to metals and is an ideal antimicrobial agent due to its high oxidizing potential. It is widely effective against microorganisms, is not deactivated by bacterial catalase or peroxidases, and has better solubility in lipid materials than hydrogen peroxide. It degrades to safe and environmentally friendly waste products, such as acetic acid and hydrogen peroxide, and therefore can be used in non-rinse applications. It can be used over a wide range of temperatures (0-40° C., or 32-104° F.) and pH values (3.0-7.5) in facility cleaning processes and in hard water conditions and is unaffected by protein residues.

Aldehydes: the best known of these compounds is formaldehyde, or formalin. These compounds act by denaturing microorganisms' proteins and nucleic acids. Formalin has fallen out of use for this application, however, because it is included in the list of carcinogenic substances and preparations. The antimicrobial activity of aldehydes, basically formaldehyde and glutaraldehyde, is linked to the denaturation of proteins and nucleic acids by chemical reduction. Aldehydes are very good at destroying bacteria and microscopic fungi and have excellent virucidal activity. They are used to disinfect surfaces, appliances, and instruments.

Alcohols: the two most used alcohols are ethanol and isopropanol or isopropyl alcohol. These compounds have the advantage of low toxicity, but their limited disinfecting spectrum is a disadvantage. Their use is only advisable as bactericides. The main form of antimicrobial action of alcohols is through protein denaturation, which causes the rupture of membranes. The microbicidal action of alcohols at various concentrations has been examined in a wide variety of species, with exposure periods ranging from 10 seconds to one hour. At concentrations of 60%-80%, both ethanol and isopropanol are potent virucidal agents, inactivating almost all lipophilic virus species and many hydrophilic viruses. They have potent antifungal activity, including on yeasts. Alcohols are not recommended for sterilization of medical or surgical materials, mainly because they are unable to harm sporulated microorganisms and cannot penetrate protein-rich materials. There are no known resistances developed from exposure to ethanol. Alcohols are colorless, but volatile and flammable, so they should be stored in cool, well-ventilated environments. Furthermore, alcohols evaporate quickly, so it is difficult to keep extended periods of exposure unless materials are immersed in alcohol. They are good disinfectants but are not considered high-level disinfectants (HLD) since they do not inactivate bacterial endospores; they are intermediate-level disinfectants. They are good for disinfecting clean and dry objects (medium and low risk). Alcohols are also used as antiseptics for intact skin.

Phenolic compounds: these products were among the first used in hospital disinfection due the work of Lister, a pioneer of surgical asepsis. Different phenolic compounds form the basis of many common disinfectants, sometimes being used to replace hypochlorites. Phenolic compounds are weakly acidic aromatic organic compounds and resemble alcohols in structure. Phenol is soluble in organic solvents and slightly soluble in water at room temperature, but above 66° C. (150.8° F.) it is soluble in any proportion. Halogenated or non-halogenated arylphenols have very good bactericidal activity, but their fungicidal activity is very weak, and there are ongoing discussions about their virucidal activity. Phenol and its derivatives cause irritation to the skin, respiratory mucosa, and ocular mucosa. They have allergenic and photosensitizing effects.

Chlorine compounds: the best-known compound in this family is sodium hypochlorite. Chlorine compounds act by oxidizing the protein structures in microorganisms' cell membranes, destroying their selective permeability. The main drawback of these compounds is their corrosivity. The biocidal activity of chlorine compounds mainly arises due to their capacity to form undissociated hypochlorous acid and the release of free chlorine—for this reason, care must be taken during the preparation of chlorine disinfectants. It is postulated that free chlorine and hypochlorous acid, which are formed in the chlorine solution, produce their disinfecting effect by denaturing proteins and inhibiting enzymatic reactions vital to microorganisms. Chlorine belongs to the family of halogenated compounds, of which chlorine and iodine compounds are those most often used for their bactericidal effects. Chlorine compounds are the most widely used disinfectants at an industrial level, and their use in water treatment is unparalleled. The active ingredient, chlorine, may be present in gaseous form or as hypochlorite or chloramine solutions. Chlorine gas is two and a half times heavier than air, has an intensely unpleasant suffocating odor, and is extremely toxic. In its liquid and solid forms, it is a powerful oxidizing, bleaching and disinfecting agent. Chlorine is a chemical considered irritating for the respiratory system, mucous membranes, and skin; liquid chlorine causes severe burns upon contact with the skin and eyes. The effects are more severe at higher concentrations and with longer exposure time, resulting in eye irritation and shortness of breath; symptoms of exposure to high concentrations consist of nausea and vomiting, followed by noticeable difficulty in breathing.

Quaternary ammonium compounds: these compounds represent a family of antimicrobial compounds in which the nitrogen atom has four bonds occupied by alkyl groups of varying complexity. These compounds are soluble in water and alcohol and have surfactant properties. Their advantage is that they are not corrosive at neutral pH values. On the other hand, they generate foams, which in many cases limits their uses. Another disadvantage is that they have no sporicidal effect. Quaternary ammonium molecules disrupt the normal arrangement of the cell membrane or envelope of different infectious agents, binding irreversibly to the phospholipids and proteins of these structures, which causes changes in membrane permeability, leaks of vital cytoplasmic material and the release of various metabolites to microbial cells that interfere directly with their respiratory chain or energy metabolism. Other mechanisms of action attributed to these compounds are the inactivation of enzymes and the denaturation of proteins that are essential for microbial agents' development. The biocidal action of tertiary amines, which are found along with quaternary ammonium compounds in disinfectants, also occurs due to their interaction with plasma membranes. These compounds can cause skin and mucosal (including eye) irritation at high concentrations. On the other hand, diluted solutions usually do not cause skin irritation. In allergic people, they can cause atopic dermatitis with nasal irritation or obstructive bronchial disease, and prolonged contact with the disinfectant may cause contact dermatitis. Accidental ingestion may cause nausea, vomiting and abdominal pain.

It has been mentioned above that disinfection is the chemical process that eliminates or kills pathogens and microorganisms such as viruses, bacteria and protozoa, preventing their growth. Disinfection must take into account not only the effectiveness of the disinfectant, but also the durability in time of its effects after application. The full spectrum of disinfecting activity includes bactericidal, fungicidal, virucidal and sporicidal effects.

The effectiveness of a disinfectant is linked to its formula, the application dose, the amount applied per m², and the mode of application.

Alcohols, chlorine compounds and quaternary ammonium compounds are the most widely used chemical agents for the production of disinfectant and sanitizing products that go on the market. Numerous brands have products based on these compounds. However, these 3 compounds have weaknesses, since they can be toxic to some degree, their residual effects have a short duration, the regular operation of an establishment must be stopped for their application and they cannot be used directly on people, so they may only be applied on surfaces.

### Innovation in Disinfectants and Sanitizers:

The current pandemic has posed the challenge of applying products with greater effectiveness and residual effects, and as a result some innovations have emerged at the national and international levels.

In Chile, copper nanoparticles are being used which, when added to another compound, either a quaternary ammonium compound or alcohol (the mixtures known on the market), grants them greater effectiveness and residual effect. An example of a product with copper nanoparticles and a quaternary ammonium compound is “Decutec” (https://bit.ly/3cCNzHl). A product with copper nanoparticles and alcohol in the Chilean market is “Aircop” (https://bit.ly/3aohyAd).

These products are innovative, but they still use toxic components and are therefore ideally used only on surfaces.

Internationally, a product with “aegis microbe shield” nanotechnology has been put on offer (https://www.microban.com/es/antimicrobial-solutions/technologies/aegis-microbe-shield). This technology is a modified molecule consisting of a silane base (used in the glass industry) for fixability; an 18-carbon chain that breaks membranes, and nitrogen that gives it a positive charge (+) which attracts microbes with a negative charge on their membranes or walls and causes the electric shock that eliminates them. This product is based on a toxic component, silane, so it can also only be applied on surfaces.

Another example of innovation is a recently released Asian product, MAP-1. This is a product developed by a team of scientists from the Hong Kong University of Science and Technology (HKUST) that not only removes virus and other germs, but also creates a disinfectant coating for up to 90 days after it is applied. It thus maintains its disinfecting activity for longer, protecting surfaces from the proliferation of bacteria and viruses. This product is composed of millions of disinfectant-loaded nanocapsules surrounded by polymers that enhance the product's action when exposed to heat.

The experience with the COVID-19 pandemic worldwide had as one of its consequences the need to perform better and more frequent sanitation and disinfection in all environments. These processes currently use products already known on the market, which are meant for use on surfaces and under certain conditions could have negative effects on human health.

People need to keep spaces free of contamination and avoid contagion as much as possible. Even in this context, a large part of the population does not know the products that are used for these purposes.

Countless initiatives have emerged to fight the spread of the virus, including massive disinfections in public areas and sanitation tunnels, which have been questioned due to the risks posed to people and the possible low effectiveness of the products used.

Chilean application 201503652 discloses a translucent adhesive film comprising copper nanoparticles with antibacterial, surface-protective activity, its method of preparation and its use for protecting surfaces highly exposed to bacteria and fungi.

Document MX2015017397 refers to a process for in-situ synthesis of copper nanoparticles in chitosan-polyvinyl alcohol hydrogels and use of this product as an antimicrobial agent.

Application US2008147019 discloses a system of antimicrobial components containing metal nanoparticles and chitosan and/or its derivatives.

Document US2011129536 refers to nanocomposites based on metal nanoparticles stabilized with branched polysaccharides.

### SUMMARY

The state of the art described above differs from the present solitude, since none of these documents describes a product (composition) that is highly effective as a disinfectant and sanitizer and able to be applied for daily use in environments while people carry out their activities.

The disinfectant and sanitizing composition in question is a mixture of natural components which by themselves already present proven antimicrobial effects. This invention combines the qualities of biopolymers, such as chitosan (a natural biopolymer obtained from chitin), and copper nanoparticles, resulting in a synergy that enhances the product's antimicrobial activity, with the advantage that it does not cause harm to people or the environment and does not require changes to the usual activities performed in the spaces where it can be applied.

Chitin (poly-N-Acetyl-Glucosamine) is the second most abundant biopolymer in nature. It is found in the shell of all crustaceans (crabs, prawns, shrimp, king crabs, lobsters, and krill) and also in the cell wall of insects, worms and fungi. Depending on the species, it can be found as alpha- or beta-chitin, which have different conformations.

Chitosan is a natural biopolymer obtained from chitin by chemical, electrochemical or enzymatic methods. Chemically it is a poly(D-glucosamine) and is therefore considered a biodegradable polysaccharide. Its molecular size depends on the species and age of the individuals where it is found, since they are obtained from a biosynthetic polymer.

Part of the invention involves achieving an optimal dissolution of chitosan in the minimum possible concentration of acetic acid, improving the product's acidity and therefore its safety.

The invention is a mixture of natural components which by themselves already present proven antimicrobial effects. This invention combines the qualities of oligosaccharide biopolymers, such as chitosan, and copper nanoparticles, resulting in a synergy that enhances the product's antimicrobial activity, with the advantage that it does not cause harm to people or the environment and does not require changes to the usual activities performed in the spaces where it can be applied. The product forms a protective transparent coating film on the treated surface. Microorganisms die quickly upon contact with the coated surface, since the effects of the product and of the protective barrier it creates break microbes' capsules and their cell membranes, inhibiting microbial growth on treated surfaces. This makes the product an effective disinfectant and sanitizer for a wide range of uses.

### BRIEF DESCRIPTION OF THE DRAWINGS

FIG. 1: Represents the mixture of the chitosan biopolymer with the copper nanoparticles, resulting in a disinfectant product with a synergy of both compounds which forms a film on treated surfaces.

FIG. 2: Represents the contact of microbes with the surface coated with the product (protective film).

FIG. 3: Represents the structure of a microbe with its parts affected by the product (zoomed-in view).

FIG. 4: Represents a product particle entering a microbe.

FIG. 5: Represents how a product particle affects the microbial capsule.

FIG. 6: Represents how a product particle affects the microbial membrane.

FIG. 7: Represents how a product particle alters a microbe's metabolism.

### DETAILED DESCRIPTION

### Disinfectant Evaluation Test

This test was conducted by WSS Testing and Certification.

The present test determined bactericidal activity and behavior over time in contact with smooth and rough surfaces previously contaminated with bacteria.

The following strains were used for this evaluation:
Escherichia coli (Gram −) ATCC 25922
Staphylococcus aureus (Gram +) ATCC 25923
Candida albicans (Gram +) ATCC 10231

To determine bactericidal activity, samples were taken from surfaces contaminated with different concentrations of the bacteria under study and different concentrations of the product for application on these surfaces.

Sampling was performed at certain time intervals (days) to evaluate the development or inhibition of bacterial growth over time in contact with different dilutions of the products applied.

### 1.—Procedure

### 1.1.—Preparation of Microbial Suspension

For each measurement, microbial suspensions were prepared and had their concentration determined by comparison with a turbidity standard, which allowed an estimate of the microbial suspension's density.

A 1.0 McFarland Scale tube was used as the turbidity standard. The McFarland scale is a series of different turbidity patterns, each with estimated concentrations of bacterial density.

The density of each bacterial suspension was compared with an ampoule of known turbidity (for this case, standard tube No. 1 corresponds to an estimated bacterial concentration on the order of 3.0×108/ml). By matching the pattern's turbidity with each pathogen's suspension, it can be estimated that the bacterial density is similar to the known turbidity, and thus an initial “known” count can be obtained. This makes it possible to subsequently evaluate bacterial growth or inhibition after a given time, exposing these preparations to contact with the products in solution (at different concentrations) to determine if the product's bactericidal activity remains over time on surfaces.

To this end, after each bacterial suspension matched the turbidity of tube No. 1 of the McFarland scale (3.0×108/ml), successive dilutions (107, 106, 105, 104, 103 . . . ) were prepared. After repetitions to evaluate how to best expose the results of the analysis, the decision was taken to work with the following concentrations of microbial contamination for the strains included in the study:
Escherichia coli, concentration in the order of 105
Staphylococcus aureus, concentration in the order of 105
Candida albicans, concentration in the order of 106.

### 1.2.—Contamination in Smooth and Rough Surfaces

After obtaining the bacterial suspensions detailed above, 5 smooth surfaces and 5 rough surfaces were contaminated with each strain. The five smooth and rough surfaces were prepared as follows, based on the product evaluated and the client's requirements regarding its dilutions:

| | | COMPOSITION OF |
|---|---|---|
| DILUTIONS | | THE INVENTION |
| | 1:04 | X |
| [symbol] | 1:05 | X |
| [symbol] | 1:10 | X |
| [symbol] | 1:15 | X |
| [symbol] | 1:20 | X |

[symbol]

indicates data missing or illegible when filed

After contamination of the surfaces with the strains in their indicated concentrations, the product is applied in the dilutions described in the table above.

### 1.3.—Sampling and Evaluation

After the product (disinfectant and/or sanitizer) was applied to the smooth and rough surfaces, each surface was sampled for analysis using dry swabs.

This analysis corresponds to day “0” of sampling.

After swab samples of each surface were taken, analysis of each bacterial strain was carried out using selective culture media appropriate for each target microorganism.

Seeding for each bacterial strain was performed as follows:
Escherichia coli: for this strain, E. coli and coliform count plates (3M) were inoculated with 1 ml of sample, with results obtained (colony counting) after 48 hrs. of incubation at 35° C. (95° F.).
Staphylococcus aureus: for this strain, Petri dishes with Baird-Parker agar were inoculated with 0.1 ml of sample (surface seeding), with results obtained (colony counting) after 48 hrs. of incubation at 35° C. (95° F.).
Candida albicans: seeding for this strain used Yeast-Glucose-Chloramphenicol (YGC) agar inoculated with 1 ml of sample (deep seeding), with results obtained (colony counting) after 5 days of incubation at 24° C., or 75.2° F. (as indicated by the reference standard used).

The procedure described above (seeding) was repeated on days 3, 7 and 10 of contact of the product with the smooth and rough surfaces.

### 1.4.—Evaluated Product Concentrations

The product tested is identified as:
Disinfectant and/or sanitizing composition of the invention at 2.5%

### 2.—Results

### 2.1.—Colony Counts

After the specified times and temperatures of incubation of the plates with selective agar media, the colonies in each plate were counted, producing the following results:

Note 1. The results obtained below refer to the direct count of colonies on each plate in 1 ml of sample for each case presented, without further calculations, i.e., the actual number of colonies counted on each plate was the value recorded for each day, for ease of observation and comparison.

Note 2. The treatment described as Control (+) corresponds to colony counts on surfaces seeded with bacterial strain suspensions to which no disinfectant was applied.

*TNTC: Plates with too many colonies; the expression “TNTC” (too numerous to count) refers to plates in which the development of colonies is such that a precise count of the number of colonies cannot be done.

TABLE 2
Summary of results obtained with Staphylococcus aureus
strain at days: 0, 3, 7 and 10.

| | STAPHILOCOCCUS AUREUS COUNTS - | | | | |
|---|---|---|---|---|---|
| | SMOOTH SURFACE | | | | |
| | | DAY | DAY | DAY | DAY |
| | DILUTIONS | 0 | 3 | 7 | 10 |
| PRODUCT | 1:04 | 259 | 0 | 0 | 0 |
| OF THE | 1:05 | 275 | 0 | 0 | 0 |
| INVENTION | 1:10 | 65 | 0 | 0 | 0 |
| | 1:15 | 28 | 0 | 0 | 0 |
| | 1:20 | 23 | 0 | 0 | 0 |

| | | | | |
|---|---|---|---|---|
| CONTROL (+) | 11970 | 1110 | 0 | 0 |

TABLE 3
Summary of results obtained with Candida albicans
strain at days: 0, 3, 7 and 10.

| | CANDIDA ALBICANS COUNTS - | | | | |
|---|---|---|---|---|---|
| | SMOOTH SURFACE | | | | |
| | | DAY | DAY | DAY | DAY |
| | DILUTIONS | 0 | 3 | 7 | 10 |
| PRODUCT | 1:04 | 0 | 0 | 0 | 0 |
| OF THE | 1:05 | 0 | 0 | 0 | 0 |
| INVENTION | 1:10 | 0 | 0 | 0 | 0 |
| | 1:15 | 0 | 0 | 0 | 0 |
| | 1:20 | 0 | 0 | 0 | 0 |

| | | | | |
|---|---|---|---|---|
| CONTROL (+) | 20160 | 0 | 0 | 0 |

*TNTC: Plates with too many colonies; the expression “TNTC” (too numerous to count) refers to plates in which the development of colonies is such that a precise count of the number of colonies cannot be done.

### 2.1.2.—Rough Surfaces

TABLE 4
Summary of results obtained with Escherichia coli
strain at days: 0, 3, 7 and 10.

| | ESCHERICHIA COLI COUNTS - | | | | |
|---|---|---|---|---|---|
| | ROUGH SURFACE | | | | |
| | | DAY | DAY | DAY | DAY |
| | DILUTIONS | 0 | 3 | 7 | 10 |
| PRODUCT | 1:04 | 18 | 0 | 0 | 0 |
| OF THE | 1:05 | 12 | 0 | 0 | 0 |
| INVENTION | 1:10 | 36 | 0 | 0 | 0 |
| | 1:15 | 18 | 0 | 0 | 0 |
| | 1:20 | 16 | 0 | 0 | 0 |

| | | | | |
|---|---|---|---|---|
| CONTROL (+) | TNTC | 0 | 0 | 0 |

*TNTC: Plates with too many colonies; the expression “TNTC” (too numerous to count) refers to plates in which the development of colonies is such that a precise count of the number of colonies cannot be done.

TABLE 5
Summary of results obtained with Staphylococcus aureus
strain at days: 0, 3, 7 and 10.

| | STAPHILOCOCCUS AUREUS COUNTS - | | | | |
|---|---|---|---|---|---|
| | ROUGH SURFACE | | | | |
| | | DAY | DAY | DAY | DAY |
| | DILUTIONS | 0 | 3 | 7 | 10 |
| PRODUCT | 1:04 | 1 | 0 | 0 | 0 |
| OF THE | 1:05 | 10 | 2 | 0 | 0 |
| INVENTION | 1:10 | 10 | 0 | 1 | 0 |
| | 1:15 | 18 | 0 | 1 | 0 |
| | 1:20 | 10 | 0 | 0 | 0 |

| | | | | |
|---|---|---|---|---|
| CONTROL (+) | 3400 | 850 | 119 | 0 |

TABLE 6
Summary of results obtained with Candida albicans
strain at days: 0, 3, 7 and 10.

| | CANDIDA ALBICANS COUNTS - | | | | |
|---|---|---|---|---|---|
| | ROUGH SURFACE | | | | |
| | | DAY | DAY | DAY | DAY |
| | DILUTIONS | 0 | 3 | 7 | 10 |
| PRODUCT | 1:04 | 0 | 0 | 0 | 0 |
| OF THE | 1:05 | 0 | 0 | 0 | 0 |
| INVENTION | 1:10 | 0 | 0 | 0 | 0 |
| | 1:15 | 0 | 0 | 0 | 0 |
| | 1:20 | 0 | 0 | 0 | 0 |

| | | | | |
|---|---|---|---|---|
| CONTROL (+) | 6080 | 0 | 0 | 0 |

### 3.—Evaluation of the Results

Application of the Product and its Different Concentrations in Escherichia coli Strains
For the product's application on the Escherichia coli strain, high inhibition is observed on day “zero” of contact with the product, as can be seen by comparing counts with those obtained in the same day for the Control (+) treatment. This is observed for both smooth and rough surfaces.
In rough surfaces, it should be noted that, at day “zero”, the product of the invention (Olicopper) shows higher inhibition in comparison to the two other two bacteria's evaluation.
It should also be mentioned that, based on the results observed during the evaluation test, the E. coli strain does not survive to days 3, 7 and 10 in the evaluated surfaces, since no colony development is observed in these days even in the Control (+) sample that was not in contact with the evaluated product. Therefore, no conclusion can be reached regarding the product's activity when in contact with this strain for days 3, 7 and 10, since no colony development occurred in the tests carried out during these days.
   Application of the Product on Staphylococcus aureus Strain
For the product's application on the Staphylococcus aureus strain, high inhibition of colonies at day “zero” is observed for smooth and rough surfaces. This inhibition remains as days go by, as colony development is still observed on the surfaces inoculated with the Control (+) sample in all cases up to day 7 of analysis, allowing a comparison of the analyzed product's bactericidal action up to this day.
Note that the colony development results observed show no major differences between the dilutions made to the product, since all dilutions successfully inhibited the initial bacterial load on all days of evaluation. The dilutions made to the product show no important differences in that they all show high inhibition in comparison to the positive controls, but no large differences are observed between the dilutions made to the product.
   Application of the Product on Candida albicans Strain
The product's application on the Candida albicans strain shows a behavior similar to that seen with the Escherichia coli strain, as the Control (+) samples do not remain viable after day “zero” in all cases, both on smooth and rough surfaces.
It is observed, however, that inhibition does occur with the product under evaluation on both surface types (smooth and rough) at day “zero” in comparison to colony development in the positive control treatment, and the product of the invention stands out since complete inhibition of colony growth on the plates occurred on both surface types in all the evaluated dilutions of the product.
Based on the evaluation above, it can be concluded that the product presented bactericidal action against Escherichia coli, Staphylococcus aureus and Candida albicans strains in all cases, on both evaluated surfaces (smooth and rough). However, the behavior of some strains, due to their particular viability characteristics, which differ between species and have to do with how favorable for development environments are (i.e. nutrient availability), prevented survival on inert surfaces without nutrients as days passed; likewise, strains that were initially inhibited did not grow back on later days. The Staphylococcus aureus strain was an exception, as it did remain on the surfaces and therefore could be evaluated and compared after day zero.

TABLE 7
Comparative table of products vs duration
of their disinfectant effectiveness

| Compounds | Duration of effectiveness | |
|---|---|---|
| Sodium hypochlorite | 1 | day |
| Quaternary ammonium compound | 1 | day |
| Quaternary ammonium compound with alcohol | 1 | day |
| Quaternary ammonium compound with copper | 15 | days |
| nanoparticles | | |
| Chitosan with copper nanoparticles (invention) | 20-30 | days |

TABLE 8
Comparative table of products vs corrosivity

| Compounds | Corrosivity |
|---|---|
| Sodium hypochlorite | pH centered at 11, irritant |
| Quaternary ammonium compound | pH centered between 12 and 13 |
| Quaternary ammonium compound with | pH centered between 12 and 13 |
| alcohol | |
| Quaternary ammonium with copper | pH centered between 12 and 13 |
| nanoparticles | |
| Chitosan with copper nanoparticles | pH centered between 6 and 7 |
| (invention) | |

Note:
The invention is less corrosive at pH values close to 7

Note: The invention is less corrosive at pH values close to 7

TABLE 9
Comparative table of products regarding their filmogenic properties

| Compounds | Filmogenic properties |
|---|---|
| Sodium hypochlorite | No |
| Quaternary ammonium compound | No |
| Quaternary ammonium compound with alcohol | No |
| Quaternary ammonium with copper nanoparticles | No |
| Chitosan with copper nanoparticles (invention) | Yes |

TABLE 10
Comparative table of the products' stability

| Compounds | Stability |
|---|---|
| Sodium hypochlorite | Not stable, chlorine solutions |
| | should be prepared daily |
| Quaternary ammonium compound | Stable at room temperature and |
| | protected from exposure to light |
| Quaternary ammonium compound with | Stable at room temperature and |
| alcohol | protected from exposure to light |
| Quaternary ammonium with copper | Stable at room temperature and |
| nanoparticles | protected from exposure to light |
| Chitosan with copper nanoparticles | Stable without restrictions |
| (invention) | |

TABLE 11
Comparative table of products' compatibility with detergents

| Compounds | Compatibility with detergents |
|---|---|
| Sodium hypochlorite | Not compatible. Sodium hypochlorite |
| | loses its effectiveness if mixed |
| | with detergents |
| Quaternary ammonium compound | Compatible. |
| Quaternary ammonium compound | Compatible. |
| with alcohol | |
| Quaternary ammonium with copper | Compatible. |
| nanoparticles | |
| Chitosan with copper nanoparticles | Compatible. |
| (invention) | |

TABLE 12
Comparative table of hazards involved when handling the products.

| Compounds | Handling hazards (adverse effects) |
|---|---|
| Sodium hypochlorite | Liquid chlorine causes severe burns |
| | on contact with skin and eyes. |
| | Exposure to high concentrations can |
| | cause nausea and vomiting. |
| Quaternary ammonium compound | May cause skin and mucosal irritation |
| | at high concentrations. |
| Quaternary ammonium compound | May cause skin and mucosal irritation |
| with alcohol | at high concentrations. |
| Quaternary ammonium with copper | May cause skin and mucosal irritation |
| nanoparticles | at high concentrations. |
| Chitosan with copper nanoparticles | No adverse effects. |
| (invention) | |

TABLE 13
Comparative table of the application of
the products in the presence of people

| | Application in the presence |
|---|---|
| Compounds | of people |
| Sodium hypochlorite | No and ventilation recommended |
| Quaternary ammonium compound | No and ventilation recommended |
| Quaternary ammonium compound | No and ventilation recommended |
| with alcohol | |
| Quaternary ammonium with copper | No and ventilation recommended |
| nanoparticles | |
| Chitosan with copper nanoparticles | Yes and no need to ventilate |
| (invention) | |

Tables 7 to 13 show comparisons of state-of-the-art products with the disinfectant and sanitizing composition of the present application. showing greater advantage of the product of the present invention, as a disinfectant and sanitizer, than the products disclosed in the prior state-of-the-art.

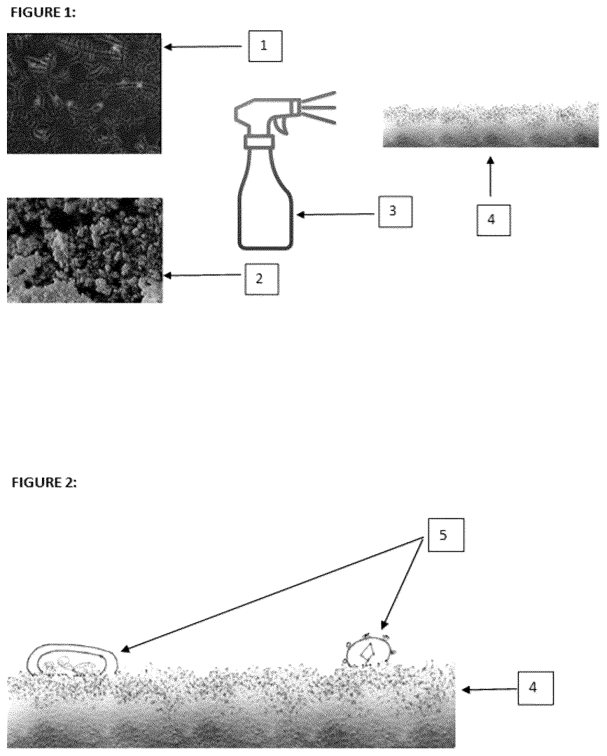

FIG. 1 shows the liquid disinfectant and sanitizing composition of organic origin (3) corresponding to the invention, which has a wide spectrum of activity and prolonged effect, is completely harmless to people due to its zero toxicity and does not generate side effects on surfaces or to the environment. What maximizes the product's antimicrobial potential is a mix of an oligosaccharide biopolymer, such as chitosan (1), with copper nanoparticles (2).

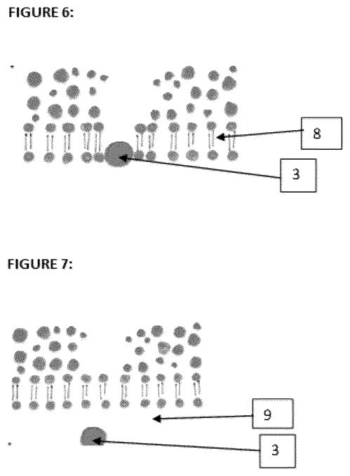

The composition (3) forms a transparent protective coating film (4) on the treated surface. Microorganisms die quickly upon contact with the coated surface (5), since the effects of the product and of the protective barrier it creates break microbes' capsules (7) and their cell membranes (8), inhibiting microbial growth on treated surfaces. This makes the product an effective disinfectant and sanitizer for a wide range of uses (see FIGS. 2, 3, 4, 5, 6 and 7).

The efficiency in antimicrobial and antiviral activity occurs due to adherence of viruses and bacteria to the surface treated with the product, where they are eliminated in step (5) (see FIG. 2).

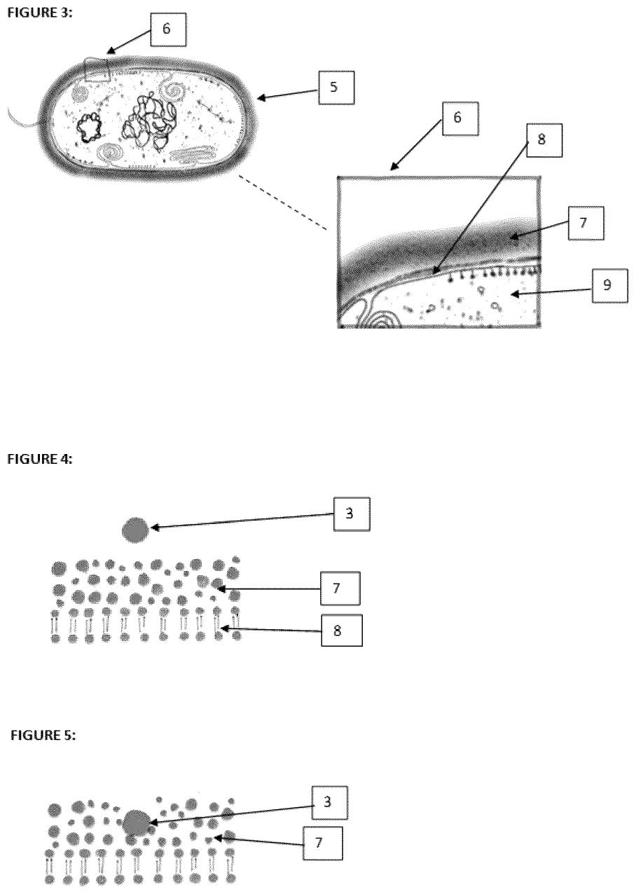

The product's antimicrobial activity is explained by the ability of the oligosaccharides in its composition to alter the permeability of bacterial cell membranes (steps 7 and 8), preventing the correct flow of nutrients (9) and leading to bacterial death; the product's effects also cause a sequestration of essential nutrients that leads to microbial cell death. Finally, antimicrobial activity is also produced by penetration of the product's molecules into cells, inhibiting protein synthesis and the function of intracellular enzymes (9) (see FIGS. 3 and 7).

The copper nanoparticles (2) increase the product's antimicrobial activity. For viruses, particularly, the role of the copper nanoparticles is to alter enzymatic molecular structures and their functions (6). The mechanism of action is based on inactivating the activity of the enzyme protease (which plays an essential role in viral replication). Moreover, copper nanoparticles cause significant damage to the phospholipid envelopes of viruses (see FIG. 3).

In summary, this application describes a disinfectant and sanitizing composition comprising a mixture of 1-4% w/v of a chitin derivative, such as chitosan, with molecular weight between 300.000 gr/mol and 600.000 gr/mol and viscosity between 48 and 54 mPa/s, 1-3% w/w of an organic acid, 0.002-0.01% w/v of copper nanoparticles, 1-2% v/v of a vegetable essential oil, and the remainder being water. The chitosan mixture of the composition described has an acetylation degree higher than 70%.

The chitosan mixture preferably used is poly(beta-(1,4)-2-amino-2-deoxy-D-glucose) and poly(beta-(1,4)-D-glucosamine).

The disinfectant and sanitizing composition has a pH between 6-7.

The size of the copper nanoparticles used is between 20 nm and 100 nm.

The organic acid used may be acetic acid, malic acid, lactic acid or citric acid, preferably acetic acid; and the organic oil used may be lavender (Lavandula angustifolia oil) and/or tea tree oil (Malaleuca alternifolia leaf oil), preferably lavender (Lavandula angustifolia oil).

The disinfectant and sanitizing composition is harmless to people. The disinfectant composition in question can be diluted up to 20 times its volume (1:20), and therefore can be atomized using different cold atomization technologies.

Also disclosed is a method to prepare the disinfectant and sanitizing composition, comprising the following steps:
a) obtain a solution containing a mixture of 1-4% w/v chitosan dissolved in a 2-3% v/v acetic acid solution;
b) separately, prepare a dispersion of copper nanoparticles in alcohol by dispersing the copper nanoparticles in alcohol including lavender oil and strongly stirring to produce the dispersion;
c) use an infusion pump to slowly mix the solutions obtained from steps (a) and (b);
d) filter the mixture obtained in step (c);
e) disperse the filtered suspension obtained in step (d) by stirring it with a recirculation pump;
f) adjust the pH to a range of 6-7;
g) obtain the desired disinfectant composition.

The disinfectant and sanitizing composition described can be used for disinfection and general sanitation of utensils, furniture, equipment, walls, and floor in various environments. Given its non-harmful effect on people, the product can be used in sanitation tunnels.

Furthermore, the disinfectant and sanitizing composition serves as an environmental deodorizer.

### Advantages of the Disinfectant and Sanitizing Composition of the Present Invention:

The product is a mixture with a broad spectrum of bactericidal, fungicidal and virucidal activity;
It exhibits antimicrobial activity against a broad spectrum of pathogenic microorganisms after short contact times;
The product is stable in the long term, as it does not evaporate easily;
It has high residual power (duration of effectiveness);
Its use and storage pose no risks;
It does not emit toxic elements in its decomposition;
The product is not toxic and handling it does not cause irritation;
It is filmogenic, i.e. creates a protective film;
It is a biocompatible product and does not cause harm to people; it can even be applied directly on skin;
It is a biodegradable product;
It can be used in indoor and outdoor environments and in sanitation tunnels;
While both components of the mixture are used by themselves as antibacterials, no existing products mix both for disinfection and sanitization of areas and people.

1. A disinfectant and sanitizing composition, comprising a mixture of 1-4% w/v of a chitin derivative, such as the biopolymer chitosan, with molecular weight between 300.000 gr/mol and 600.000 gr/mol and viscosity between 48 and 54 mPa/s, 1-3% w/w of an organic acid, 0.002-0.01% w/v of copper nanoparticles, 1-2% v/v of a vegetable essential oil, and the remainder being water.

2. The disinfectant and sanitizing composition of claim 1, wherein use of the chitosan mixture is poly(beta-(1,4)-2-amino-2-deoxy-D-glucose) and poly(beta-(1,4)-D-glucosamine).

3. The disinfectant and sanitizing composition of claim 1, wherein a chitosan acetylation degree higher than 70%.

4. The disinfectant and sanitizing composition of claim 1, wherein a pH between 6 and 7.

5. The disinfectant and sanitizing composition of claim 1, wherein copper nanoparticles with a size between 20 nm and 100 nm.

6. The disinfectant and sanitizing composition of claim 1, wherein the organic acid used includes any one more of acetic acid, malic acid, lactic acid or citric acid.

7. The disinfectant and sanitizing composition of claim 6, wherein the organic acid used is acetic acid.

8. The disinfectant and sanitizing composition of claim 1, wherein the essential oil used includes any one or more of lavender oil (Lavandula angustifolia oil) or tea tree oil (Malaleuca alternifolia leaf oil).

9. The disinfectant and sanitizing composition of claim 8, wherein the essential oil used is lavender oil (Lavandula angustifolia oil).

10. The disinfectant and sanitizing composition of claim 9, wherein the composition is harmless to people.

11. The disinfectant and sanitizing composition of claim 10, wherein the composition is diluted up to 20 times its volume (1:20) and therefore is adapted to be atomized using different cold atomization technologies.

12. A method for preparing a disinfectant and sanitizing composition having a mixture of 1-4% w/v of a chitin derivative such as the biopolymer chitosan, with molecular weight between 300.000 gr/mol and 600.000 gr/mol and viscosity between 48 and 54 mPa/s, 1-3% w/w of an organic acid, 0.002-0.01% w/v of copper nanoparticles, 1-2% v/v of a vegetable essential oil, and the remainder being water, the method comprising:
obtaining a solution containing a mixture of 1-4% w/v chitosan dissolved in a 2-3% v/v acetic acid solution;
separately, preparing a dispersion of copper nanoparticles in alcohol by dispersing the copper nanoparticles in alcohol including lavender oil and strongly stirring to produce the dispersion;
using an infusion pump to slowly mix the solutions obtained from steps (a) and (b);
filtering the mixture obtained in step (c);
dispersing the filtered suspension obtained in step (d) by stirring it with a recirculation pump;
adjusting the pH to a range of 6-7;
obtaining the desired disinfectant and sanitizing composition.

13. A use of a disinfectant and sanitizing composition having a mixture of 1-4% w/v of a chitin derivative, such as the biopolymer chitosan, with molecular weight between 300.000 gr/mol and 600.000 gr/mol and viscosity between 48 and 54 mPa/s, 1-3% w/w of an organic acid, 0.002-0.01% w/v of copper nanoparticles, 1-2% v/v of a vegetable essential oil, and the remainder being water, wherein the composition is adapted for the disinfection and general sanitation of utensils, furniture, equipment, walls and floor in various environments.

14. A use of a disinfectant and sanitizing composition having a mixture of 1-4% w/v of a chitin derivative, such as the biopolymer chitosan, with molecular weight between 300.000 gr/mol and 600.000 gr/mol and viscosity between 48 and 54 mPa/s, 1-3% w/w of an organic acid, 0.002-0.01% w/v of copper nanoparticles, 1-2% v/v of a vegetable essential oil, and the remainder being water, wherein, due to its non-harmful effect on people, is adapted for use with sanitation tunnels.

15. The disinfectant and sanitizing composition of claim 1, wherein the composition serves as an environmental deodorizer.
